(19) United States
(12) Patent Application Publication
(10) Pub. No.: US 2012/0136292 A1
(43) Pub. Date: May 31, 2012

(54) Non-Invasive Cervical-Thoracic Correction System

(76) Inventor: David E. J. Pepin, Grant Park, IL (US)

(21) Appl. No.: 12/711,972

(22) Filed: Feb. 24, 2010

Related U.S. Application Data
(60) Provisional application No. 61/155,190, filed on Feb. 25, 2009.

(51) Int. Cl. A61F 5/00

(52) U.S. Cl. 602/18

(57) ABSTRACT

A non-invasive cervical-thoracic correction system for therapeutic use and for correction of abnormalities in a person's posture is disclosed. The system comprises a chest brace that includes shoulder supports that fit to the person's shoulders; a body harness connected to the chest brace by way of one or more support straps so as to provide a stable foundation when fitted to the person; and a forehead pad mounted to the chest brace by way of a spring connection that provides a variable amount of tension to the forehead pads such that the person's head is biased into a position that is in keeping with normal posture. The spring connection includes a helix spring. The forehead pad is mounted to the chest brace by way of an adjustable joint that allows the person to adjust a force providing the bias to the head.

### RELATED APPLICATION

This application is related to and claims priority to U.S. Provisional Application No. 61/155,190, filed Feb. 25, 2009, entitled “Non-Invasive Cervical-Thoracic Correction System,” which application is hereby incorporated by reference in its entirety for all that is expressly or inherently teaches, without exclusion of any portion thereof.

### BRIEF DESCRIPTION OF THE SEVERAL VIEWS OF THE DRAWING(S)

FIG. 1 is a photograph of a prototype of the inventive non-invasive cervical-thoracic correction system, which includes a forehead pad, a helix spring, a chest brace, chest support straps and a body harness;

FIG. 2 illustrates the system of the invention fitted to a person;

FIG. 3 illustrates the system fitted to a person and showing how the system fits over the persons shoulders and around the person's torso;

FIG. 4 is a line drawings of the system in perspective;

FIG. 5 is an enlarge photographic view of the adjustments in the system that provide for variable tension or bias to the person's forehead, showing that each of the two arms of forehead pad includes tow adjustment points;

FIG. 6 is a photograph a showing both arms of the forehead pad that connect to the chest brace portion of the system by way of the several adjustment points;

FIG. 7 is a view of the system looking down from the top of the system; and

FIG. 8 is a diagram illustrating the helix force imparted by the helix spring in the system.

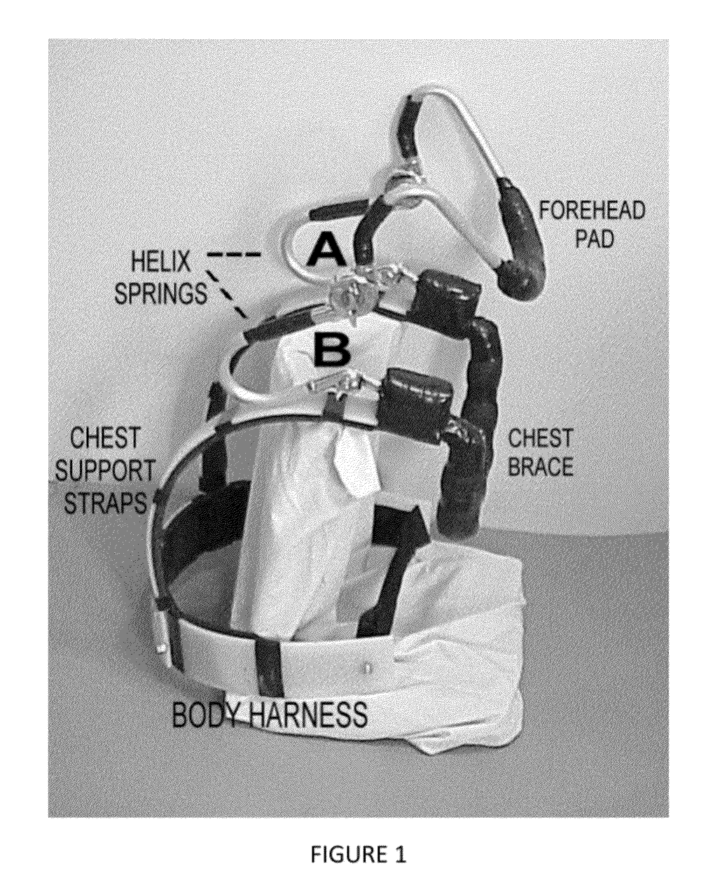

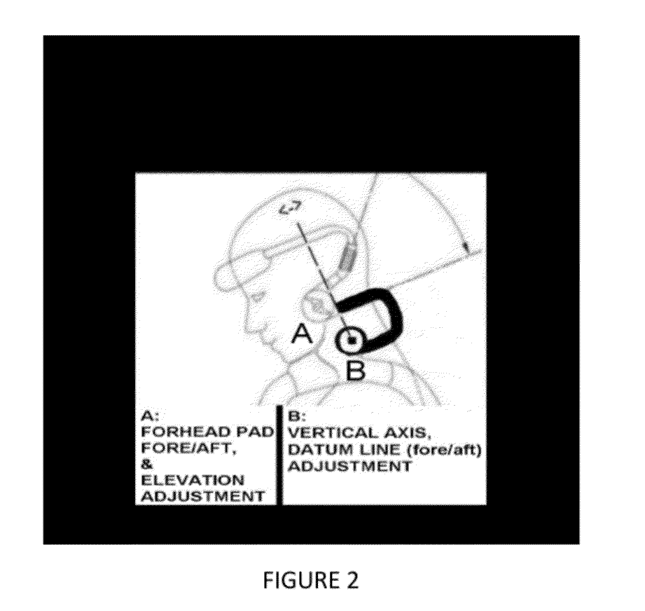

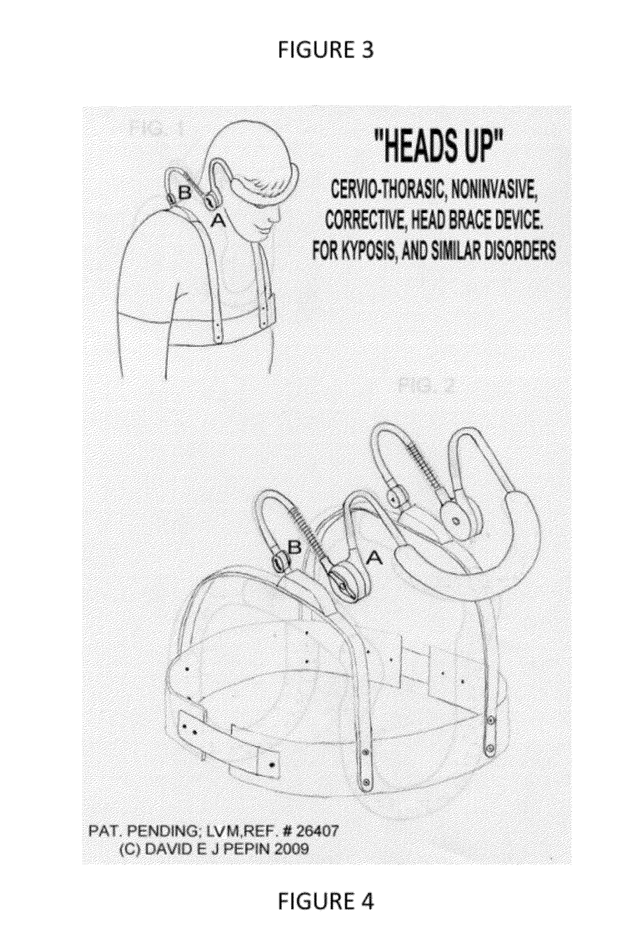

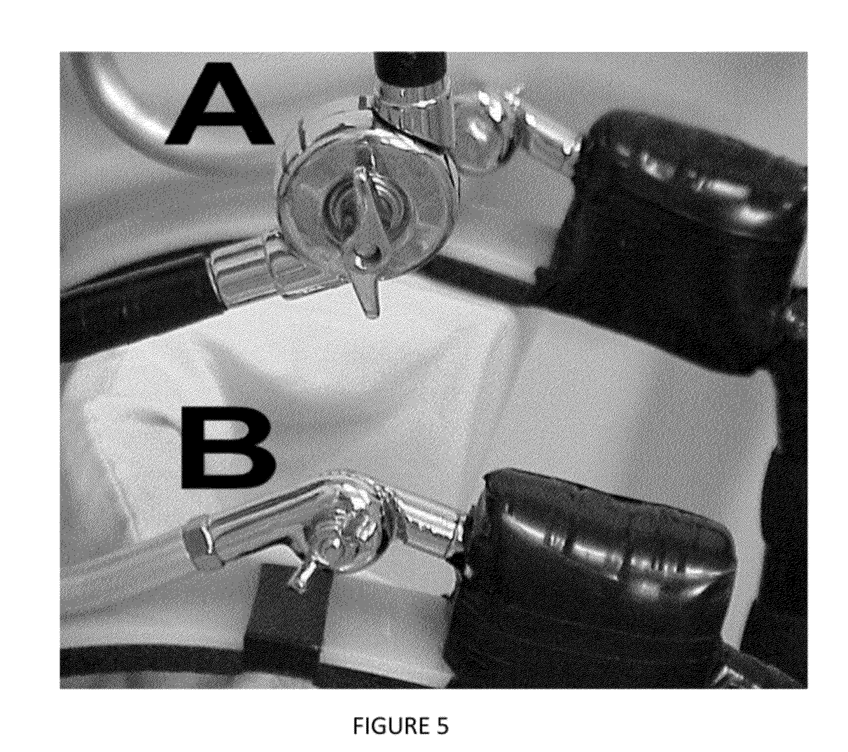

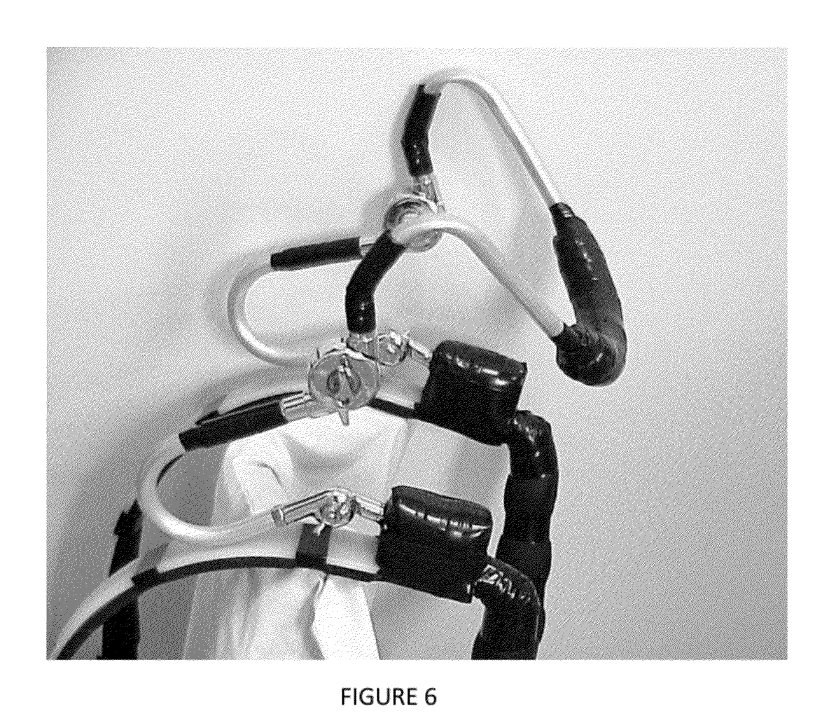

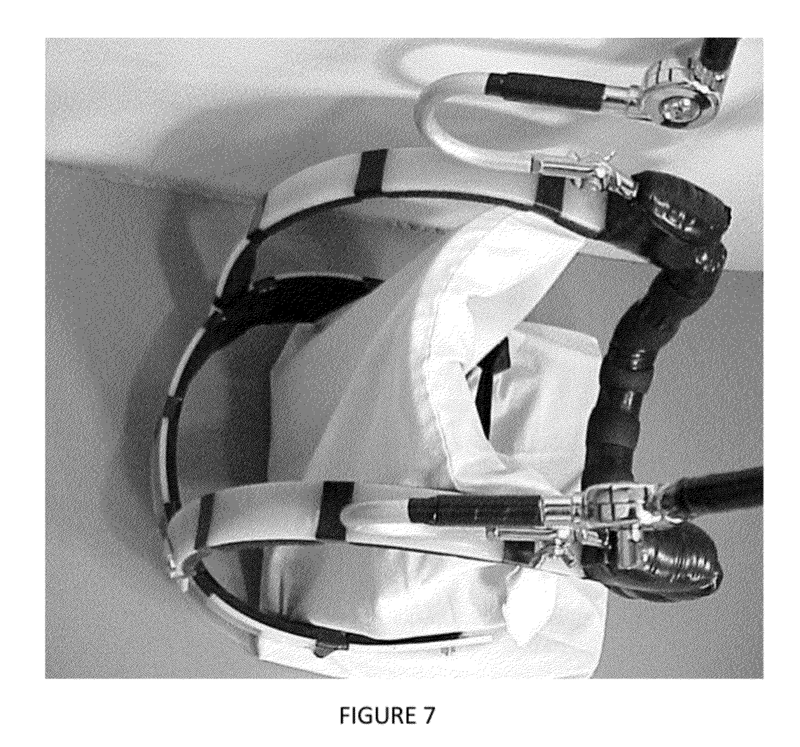

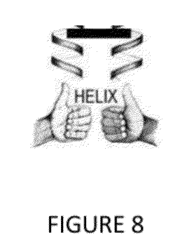

### SUMMARY OF THE INVENTION

A non-invasive cervical-thoracic correction system for therapeutic use and for correction of abnormalities in a person's posture is disclosed. The system comprises a chest brace that includes shoulder supports that fit to the person's shoulders; a body harness connected to the chest brace by way of one or more support straps so as to provide a stable foundation when fitted to the person; and a forehead pad mounted to the chest brace by way of a spring connection that provides a variable amount of tension to the forehead pads such that the person's head is biased into a position that is in keeping with normal posture.

The spring connection includes a helix spring.

The forehead pad is mounted to the chest brace by way of an adjustable joint that allows the person to adjust a force providing the bias to the head.

### DETAILED DESCRIPTION OF THE INVENTION

The Heads Up Head/Neck collar or brace is a noninvasive cervical-thoracic corrective system for conditions such as kyphosis, degenerative joint disease, complications resulting from C-Spine fractures, osteoporosis and similar conditions. It functions by utilizing the pressure forces exerted by the head and cervical-thoracic region in the opposing direction of the curvature. No additional traction or other forces are added. This is accomplished by utilizing the natural or inherent neutral balancing forces of the helix. This prevents aggravation or premature advancement of the condition. The gentle helical balancing action is in most cases corrective and therapeutic to systematic relief of pain.

Slowly and gently the Head/Neck collar or brace make corrections to the condition until the cervical position is returning resulting in the cervical spine correcting itself toward its normal position. It may maintain this position through continued therapy or permit the possibility of spinal fusion. Thus, aspects of the invention greatly reduce the high cost and the risk of undergoing cervical procedures. Helical adjustment lever A adjusts the position of the forehead pad fore and aft. Lever A also adjusts the elevation and comfort factors of the support pad. Helix adjustment lever B adjusts the vertical axis of the cervical datum line fore and aft. These functions may be adjusted by the user for progress and comfort.

The function of the body torso harness is variable and may be necessary to alter it to accommodate the needs of the individual user.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A non-invasive cervical-thoracic correction system for therapeutic use and for correction of abnormalities in a person's posture, the system comprising:
a chest brace that includes shoulder supports that fit to the person's shoulders;
a body harness connected to the chest brace by way of one or more support straps so as to provide a stable foundation when fitted to the person; and
a forehead pad mounted to the chest brace by way of a spring connection that provides a variable amount of tension to the forehead pads such that the person's head is biased into a position that is in keeping with normal posture.

2. The system of claim 1 wherein the spring connection includes a helix spring.

3. The system of claim 1 wherein the forehead pad is mounted to the chest brace by way of an adjustable joint that allows the person to adjust a force providing the bias to the head.
